(19) United States
(12) Patent Application Publication
(10) Pub. No.: US 2026/0366066 A1
(43) Pub. Date: Nov. 19, 2026

(54) STRUCTURES AND METHODS FOR AMPLIFYING POLYNUCLEOTIDES USING SACRIFICIAL MATERIALS

(71) Applicant: Illumina, Inc., San Diego, CA (US)

(72) Inventors: Tracy Fung, San Mateo, CA (US); Rean Silke Musa, La Jolla, CA (US); Vanessa Montaño-Machado, San Diego, CA (US)

(73) Assignee: Illumina, Inc., San Diego, CA (US)

(21) Appl. No.: 19/486,854

(22) PCT Filed: Oct. 16, 2024
(86) PCT No.: PCT/US2024/051524

§ 371 Date: Nov. 21, 2025

Related U.S. Application Data
(60) Provisional application No. 63/590,907, filed on Oct. 17, 2023.

(51) Int. Cl. C12Q 1/6844; C12Q 1/6874

(52) U.S. Cl. CPC C12Q 1/6844; C12Q 1/6874

(57) ABSTRACT

In some examples, a method of amplifying a polynucleotide includes capturing the polynucleotide at a recess of a sacrificial material disposed on a substrate; removing at least a portion of the sacrificial material to expand the recess having the polynucleotide captured therein; and amplifying the polynucleotide at the expanded recess. A hydrogel may be disposed within the recess and may include a capture primer to which the polynucleotide hybridizes when capturing the polynucleotide; and amplification primers to which amplicons of the polynucleotide hybridize when amplifying the polynucleotide. The hydrogel may expand from the recess into the expanded recess.

### CROSS-REFERENCE TO RELATED APPLICATIONS

This application claims the benefit of U.S. Provisional Patent Application No. 63/590,907, filed Oct. 17, 2023 and entitled “Structures and Methods for Amplifying Polynucleotides Using Sacrificial Materials,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2685-PCT.xml”, was created on Oct. 9, 2024 and is 8 KB in size.

### FIELD

This application generally relates to amplifying polynucleotides.

### BACKGROUND

Cluster amplification is an approach to amplifying polynucleotides, for example for use in genetic sequencing. Target polynucleotides are captured by primers (e.g., P5 and P7 primers) coupled to a substrate surface in a flowcell, and form “seeds” at random locations on the surface. Cycles of amplification are performed to form clusters on the surface around each seed. The clusters include copies, and complementary copies, of the seed polynucleotides. In some circumstances, the substrate is patterned so as to define regions that bound different clusters, such as wells that may be filled with respective clusters.

### SUMMARY

Examples provided herein are related to amplifying polynucleotides using sacrificial materials. Structures and methods for performing such amplification are disclosed.

Some examples herein provide a method of amplifying a polynucleotide. The method may include capturing the polynucleotide at a recess of a sacrificial material disposed on a substrate. The method may include removing at least a portion of the sacrificial material to expand the recess at which the polynucleotide is captured. The method may include amplifying the polynucleotide at the expanded recess.

In some examples, a hydrogel is disposed within the recess of the sacrificial material. The hydrogel may include a capture primer to which the polynucleotide hybridizes when capturing the polynucleotide. The hydrogel may include amplification primers to which amplicons of the polynucleotide hybridize when amplifying the polynucleotide. In some examples, the hydrogel expands from the recess into the expanded recess.

In some examples, only a portion of the sacrificial material is removed. In some examples, the sacrificial material is entirely removed.

In some examples, the sacrificial material includes silicon dioxide, silicon nitride, or a polymer.

In some examples, the sacrificial material is removed using an etchant. In some examples, the etchant includes citrate, tris(hydroxymethyl)aminomethane, carbonate buffer, photoresist developer, cleaning concentrate, TMAH (tetramethylammonium hydroxide), or DMSO (dimethylsulfoxide).

In some examples, the substrate includes a first etch stop on which the sacrificial material is disposed, the first etch stop inhibiting damage to the substrate when removing at least the portion of the sacrificial material. In some examples, the first etch stop includes tantalum oxide.

In some examples, a second etch stop is disposed on the substrate, the second etch stop substantially surrounding the recess. In some examples, the second etch stop defines a lateral area of the expanded recess. In some examples, the second etch stop includes tantalum oxide. In some examples, the second etch stop includes tantalum.

In some examples, the substrate includes at least one of a glass, a semiconductor, or a polymer.

In some examples, the substrate does not include any image sensor.

In some examples, the substrate includes an image sensor having a lateral area. In some examples, the recess has a lateral area that is less than about 50% of the lateral area of the image sensor. In some examples, the expanded recess has a lateral area that is at least about 80% of the lateral area of the image sensor. In some examples, a single recess is disposed over the image sensor. In some examples, the single recess is approximately centered over the image sensor.

In some examples, the recess has an aspect ratio of about 1:1 to about 4:1 (height:width). In some examples, the expanded recess has an aspect ratio of at least about 1:2 to about 1:100 (height:width). In some examples, the recess has a width of between about 100 nm and about 300 nm. In some examples, the expanded recess has a width of between about 500 nm and about 1000 nm.

Some examples herein provide a method for sequencing a polynucleotide. The method may include capturing and amplifying the polynucleotide using any of the aforementioned methods; and sequencing the amplified polynucleotide at the expanded recess.

Some examples herein provide a structure. The structure may include a substrate. The structure may include a sacrificial material disposed on the substrate and having a recess therein. The structure may include a hydrogel disposed within the recess of the sacrificial material. The hydrogel may include a capture primer for capturing a polynucleotide; and amplification primers for amplifying the polynucleotide. The recess may be expandable using an etchant that diffuses through the hydrogel to at least partially remove the sacrificial material, substantially without damaging the capture primer or the amplification primers.

In some examples, the hydrogel is expandable from the recess into an expanded recess that is formed by expanding the recess using the etchant.

In some examples, the sacrificial material includes silicon dioxide, silicon nitride, or a polymer.

In some examples, the sacrificial material is removable using an etchant. In some examples, the etchant includes citrate, tris(hydroxymethyl)aminomethane, carbonate buffer, photoresist developer, cleaning concentrate, TMAH (tetramethylammonium hydroxide), or DMSO (dimethylsulfoxide).

In some examples, the substrate includes a first etch stop on which the sacrificial material is disposed, the first etch stop inhibiting damage to the substrate when removing at least the portion of the sacrificial material. In some examples, the first etch stop includes tantalum oxide.

In some examples, the structure further includes a second etch stop disposed on the substrate, the second etch stop substantially surrounding the recess. In some examples, the second etch stop defines a lateral area of an expanded recess formed by the at least partial removal of the sacrificial material. In some examples, the second etch stop includes tantalum oxide. In some examples, the second etch stop includes tantalum.

In some examples, the substrate includes at least one of a glass, a semiconductor, or a polymer.

In some examples, the substrate does not include any image sensor.

In some examples, the substrate includes an image sensor having a lateral area. In some examples, the recess has a lateral area that is less than about 50% of the lateral area of the image sensor. In some examples, the expanded recess has a lateral area that is at least about 80% of the lateral area of the image sensor.

In some examples, a single recess is disposed over the image sensor. In some examples, the single recess is approximately centered over the image sensor.

In some examples, the recess has an aspect ratio of about 1:1 to about 4:1 (height:width). In some examples, the expanded recess has an aspect ratio of at least about 1:2 to about 1:100 (height:width). In some examples, the recess has a width of between about 100 nm and about 300 nm. In some examples, the expanded recess has a width of between about 500 nm and about 1000 nm.

Some examples herein provide a method for sequencing a polynucleotide. The method may include capturing and amplifying the polynucleotide using any of the aforementioned structures; and sequencing the amplified polynucleotide at an expanded recess that is formed by expanding the recess using the etchant.

Some examples herein provide a kit that includes any of the aforementioned structures; and an etchant for at least partially removing the sacrificial material.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the advantages and benefits as described herein.

### BRIEF DESCRIPTION OF DRAWINGS

FIGS. 1A-1C schematically illustrate examples of amplifying polynucleotides on a substrate.

FIGS. 2A-2L schematically illustrate example structures and operations in a process flow for amplifying a polynucleotide on a substrate using a sacrificial material.

FIGS. 3A-3D illustrate example structures that may be included in the structures described with reference to FIGS. 2A-2L.

FIG. 4 illustrates an example flow of operations in a method for amplifying a polynucleotide using a sacrificial material.

FIG. 5 illustrates etch rates measured for different materials under different conditions.

FIG. 6 illustrates etch rates measured for silicon nitride at 60 °C using different etchants.

### DETAILED DESCRIPTION

Examples provided herein are related to amplifying polynucleotides using a sacrificial material. Structures and methods for performing such amplification are disclosed.

The monoclonality of a cluster resulting from amplification of target polynucleotide(s) may affect the ease with which the target polynucleotide(s) in that cluster subsequently may be sequenced, e.g., using sequencing-by-synthesis (SBS). Polynucleotides having the same sequence as each other are considered to be “monoclonal,” while polynucleotides having different sequences are considered to be “polyclonal.” The greater the monoclonality of a cluster—that is, the greater the number of polynucleotides having the same sequence as each other in that cluster—the greater the likelihood that a signal from the in ensemble of polynucleotides within that cluster will be sufficient to correctly sequence the polynucleotide having the greatest number amplicons using SBS. A cluster with a number of amplicons of the same polynucleotide sufficient to correctly sequence that polynucleotide is considered to be “functionally monoclonal,” even if that cluster may be polyclonal. Illustratively, a cluster from which about 60% or more of the SBS signal comes from amplicons of a particular polynucleotide, and from which about 40% or less of the SBS signal comes from amplicons of one or more other polynucleotides, may be functionally monoclonal, and as such may be accurately sequenced using SBS. In some examples, the amount of SBS signal scales approximately linearly with the amount of a polynucleotide, and as such a cluster with about 60% or more of amplicons of a particular polynucleotide, and with about 40% or less of amplicons of one or more other polynucleotides, may be functionally monoclonal.

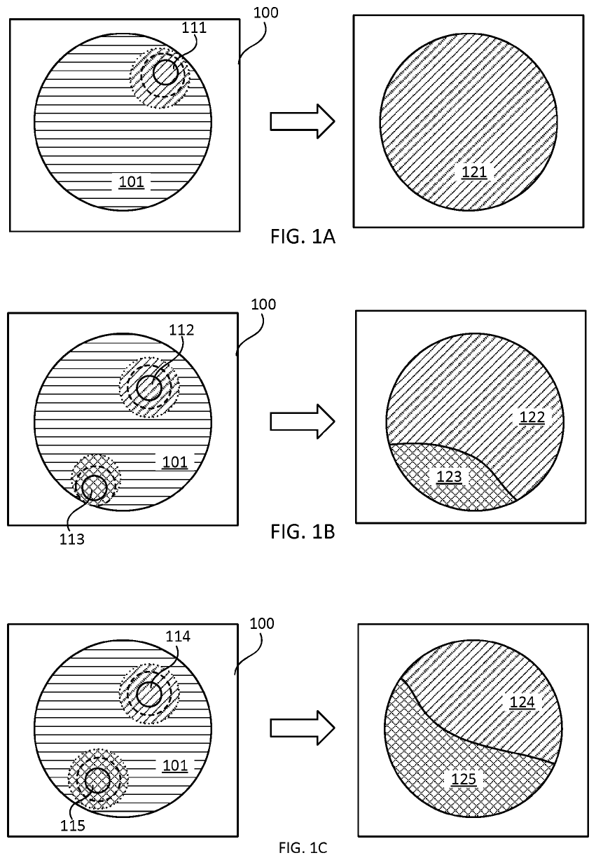

The extent to which a given cluster is monoclonal may relate to a number of factors. For example, FIGS. 1A-1C schematically illustrate examples of amplifying polynucleotides on a substrate. As shown in FIG. 1A, capture and amplification of a single seed 111 on substrate region 101 may result in monoclonal cluster 121 which may fill the substrate region, and which readily may be used for SBS (the dashed and dotted circles being intended to represent expansion of the cluster over time). In comparison, as shown in FIG. 1B, capture and amplification of two seeds 112, 113 on substrate region 101 may result in a polyclonal cluster having a first region 122 resulting from amplification of seed 112, and a second region 123 resulting from amplification of seed 113. In this example, the extent to which the cluster is polyclonal may be sufficiently low that the cluster may be useful for SBS. For example, about 60% or more of the signal from substrate region 101 may be from the amplicons of seed 112 in first region 122 and about 40% or less of the signal from substrate region 101 may be from the amplicons of seed 113 in second region 123. For example, about 60% or more of substrate region 101 may be covered by the amplicons of seed 112 in first region 122 and about 40% or less of substrate region 101 may be covered by amplicons of seed 113 in second region 123. As such the polyclonal cluster 122, 123 may be functionally monoclonal.

However, the relative locations of different seeds on the substrate may affect the extent to which the cluster is polyclonal, and the extent to which the cluster may be useable for SBS. For example, as shown in FIG. 1C, capture and amplification of two seeds 114, 115 on substrate region 101 results in a polyclonal cluster having a first region 124 resulting from amplification of seed 112, and a second region 125 resulting from amplification of seed 115, but second region 125 is larger than second region 123 in FIG. 1B because seed 115 is more centrally located than is seed 113. For example, less than about 60% of the signal from substrate region 101 may be from the amplicons of seed 114 in first region 124 and greater than about 40% of the signal from substrate region 101 may be from the amplicons of seed 115 in second region 125, and as such the polyclonal cluster 124, 125 may not be functionally monoclonal. For example, less than about 60% of substrate region 101 may be covered by amplicons of seed 114 in first region 124 and greater than about 40% of substrate region 101 may be covered by amplicons of seed 115 in second region 125. As such the polyclonal cluster 124, 125 may not be functionally monoclonal. In this example, the extent to which the cluster is polyclonal may be too high for the cluster to be useful for SBS.

The number of seeds that may be expected to land on a given substrate region may increase with the size of the substrate. Substrate patterning may further affect the extent to which clusters are polyclonal and the extent to which such clusters are useful for SBS. For example, SBS may be performed using an image sensor (such as a complementary metal oxide semiconductor (CMOS) sensor) on which a recess (e.g., nanowell) is disposed, within which a single corresponding polynucleotide cluster is formed, and the image sensor may measure signal intensity from that cluster during different SBS operations. In other examples, SBS may be performed using a glass substrate on which a recess (e.g., nanowell) is disposed, within which a single corresponding polynucleotide cluster is formed, and free space optics may be used to measure signal intensity from that cluster during different SBS operations. In both types of implementations, the larger the size of the recess, the larger the size of the cluster. The larger the size of the cluster, and the more polynucleotides within the cluster, the greater the SBS signal intensity from that cluster. However, the larger the area the polynucleotides may seed, the greater the likelihood of multiple seeding events occurring (e.g., as described with reference to FIGS. 1B and 1C), and thus the greater the likelihood of forming clusters that are too polyclonal to be useful for SBS. In examples in which an image sensor measures the SBS signal intensity, the cluster position also may affect the image sensor's effectiveness in capturing signal from the cluster. For example, signal from a cluster on the sidewall of the recess on an image sensor may couple poorly into the image sensor as compared to a cluster positioned approximately in the center of the recess.

Examples provided herein may enhance the extent to which a cluster is monoclonal (including, but not limited to, on substrates that include image sensors, or on substrates that omit image sensors) through the use of a sacrificial material. More specifically, the sacrificial material may be used to form a relatively small recess for use during polynucleotide seeding, so as to reduce the area that is accessible to the polynucleotides and thus reduce the likelihood of multiple seeding events in the recess. The sacrificial material then is partially removed or fully removed, so as to expand the size of the recess. A cluster then is generated using the expanded recess, thus providing a greater surface area for generating amplicons (and receiving signal from such amplicons during SBS) than is available before the sacrificial material is removed. Furthermore, the initial seeding event may be performed approximately in the middle of the region in which the cluster is eventually formed, thus further improving the image sensor's signal response (in examples that use an image sensor). Accordingly, the use of the sacrificial material reduces the likelihood of generating a polyclonal cluster by limiting the number of possible seeding events prior to removal of the sacrificial material, while still permitting the generation of clusters that are large enough to obtain sufficient signal for use in SBS.

First, some terms used herein will be briefly explained. Then, some example compositions and example methods for amplifying polynucleotides using sacrificial materials will be described.

### TERMS

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another. Polynucleotides that are “partially” hybridized to one another means that they have sequences that are complementary to one another, but such sequences are hybridized with one another along only a portion of their lengths to form a partial duplex. Polynucleotides with an “inability” to hybridize include those which are physically separated from one another such that an insufficient number of their bases may contact one another in a manner so as to hybridize with one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xantbine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), peptide nucleic acid (PNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide (such as PNA), branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity. In some examples, a polymerase is used in combination with another enzyme, such as a recombinase and/or a single-strand binding protein (e.g., gp32).

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer.

A “capture primer” is intended to mean a primer that is coupled to a substrate and may hybridize to an adapter of the target polynucleotide. In some cases, a capture primer that is coupled to the substrate and may hybridize to another adapter of that target polynucleotide may be referred to as an “orthogonal capture primer.” The adapters may have respective sequences that are complementary to those of capture primers to which they may hybridize. A capture primer and an orthogonal capture primer may have different and independent sequences than one another. A capture primer that may be used to hybridize to an adapter of a target polynucleotide in order to couple that polynucleotide to the substrate, but that may not be used to grow a complementary strand during an amplification process, may in some cases be referred to as a “seeding primer.” A capture primer that may be used to grow a complementary strand during an amplification process may in some cases be referred to as an “amplification primer.” In nonlimiting examples, a capture primer may be or include DNA, RNA, PNA, modified DNA, modified RNA, or modified PNA.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible.

In some examples, a substrate includes a patterned surface. A “patterned surface” refers to an arrangement of different regions in or on an exposed layer of a substrate. For example, one or more of the regions may include feature(s) that can be used to attract and/or be coupled to a surface. The feature(s) can be separated by interstitial regions where such features are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM). The process creates hydrogel regions used for sequencing that may be stable over sequencing runs with a large number of cycles. In some examples, covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA) may be used as the hydrogel material.

In particular examples, a structured substrate may be made by patterning a suitable material with wells (e.g. microwells or nanowells), coating the patterned material with a gel material (e.g., PAZAM, SFA or chemically modified variants thereof, such as the azidolyzed version of SFA (azido-SFA)) and polishing the surface of the gel coated material, for example via chemical or mechanical polishing, thereby retaining gel in the wells but removing or inactivating substantially all of the gel from the interstitial regions on the surface of the structured substrate between the wells. Primers may be attached to gel material. A solution including a plurality of target polynucleotides (e.g., a fragmented human genome or portion thereof) may then be contacted with the polished substrate such that individual target polynucleotides will seed individual wells via interactions with primers attached to the gel material; however, the target polynucleotides will not occupy the interstitial regions due to absence or inactivity of the gel material. Amplification of the target polynucleotides may be confined to the wells because absence or inactivity of gel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate. A hydrogel may be located on a substrate, such as on a region of a flow cell.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

As used herein, the term “directly” when used in reference to a layer covering the surface of a substrate is intended to mean that the layer covers the substrate's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate via covalent or non-covalent bond(s). In certain examples, covalent attachment may be used, or any other suitable attachment in which the polynucleotides remain stationary or attached to a substrate under conditions in which it is intended to use the substrate, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-S′ orientation. Alternatively, immobilization may occur by means other than base-pairing hybridization, such as covalent attachment.

As used herein, the term “array” refers to a population of substrate regions that may be differentiated from each other according to relative location. Different molecules (such as polynucleotides) that are at different regions of an array may be differentiated from each other according to the locations of the regions in the array. An individual region of an array may include one or more molecules of a particular type. For example, a substrate region may include a single target polynucleotide having a particular sequence, or a substrate region may include several polynucleotides having the same sequence (or complementary sequences thereof). The regions of an array respectively may include different features than one another on the same substrate. Exemplary features include without limitation, wells in a substrate, beads (or other particles) in or on a substrate, projections from a substrate, ridges on a substrate or channels in a substrate. The regions of an array respectively may include different regions on different substrates than each other. Different molecules attached to separate substrates may be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded” when used in reference to a polynucleotide, is intended to mean that all or substantially all of the nucleotides in the polynucleotide are hydrogen bonded to respective nucleotides in a complementary polynucleotide. A “partially” double stranded polynucleotide may have at least about 10%, at least about 25%, at least about 50%, at least about 60%, at least about 70%, at least about 80%, at least about 90% or at least about 95% of its nucleotides, but fewer than all of its nucleotides, hydrogen bonded to nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region that includes substantially only amplicons of a given polynucleotide may be referred to as “monoclonal,” while a substrate region that includes amplicons of polynucleotides having different sequences than one another may be referred to as “polyclonal.” A substrate region that includes a sufficient number of amplicons of a given polynucleotide to be used to sequence that polynucleotide maybe referred to as “functionally monoclonal.” Illustratively a substrate region in which about 60% or greater of the amplicons are of a given polynucleotide may be considered to be “functionally monoclonal.” Additionally, or alternatively, a substrate region from which about 60% or more of a signal is from amplicons of a given polynucleotide may be considered to be “functionally monoclonal.” A polyclonal region of a substrate may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target polynucleotide. The action by which a polynucleotide in solution becomes coupled to a substrate may be referred to as “capturing” the polynucleotide, or the polynucleotide “seeding” the substrate.

As used herein, the term “image sensor” is intended to refer to an electronic circuit component that generates an electrical signal in response to irradiation by photons. An image sensor may be a component of a “pixel” which includes one or more additional circuit components, such as an amplifier, that amplify or otherwise condition the electrical signal generated by the image sensor. For simplicity, however, the terms “pixel” and “image sensor” may be used interchangeably herein. An image sensor may be a component of a sensor circuit that includes a two-dimensional array of image sensors. Likewise, a pixel (which includes a corresponding image sensor) may be a component of a sensor circuit that includes a two-dimensional array of pixels. The image sensor(s) and/or pixels may be disposed within a sensor layer that may include several different circuit components, and different materials, that work together to generate respective electrical signal(s) corresponding to irradiation by photons of respective image sensor(s). The sensor circuit may include other elements, such as analog-to-digital converters (ADCs), amplifiers, and the like, that prepare the electrical signal(s) from respective image sensor(s) for use. Nonlimiting examples of image sensors include charge coupled devices (CCDs) and active-pixel sensors (CMOS sensors).

### Compositions and Methods for Amplifying Polynucleotides Using a Sacrificial Material

Examples provided herein relate to generating clusters that are substantially monoclonal. The examples herein are particularly well suited to generating clusters for use in SBS that use imaging sensors, but it should be understood that the examples are generally applicable to any type of cluster on any suitable type of substrate.

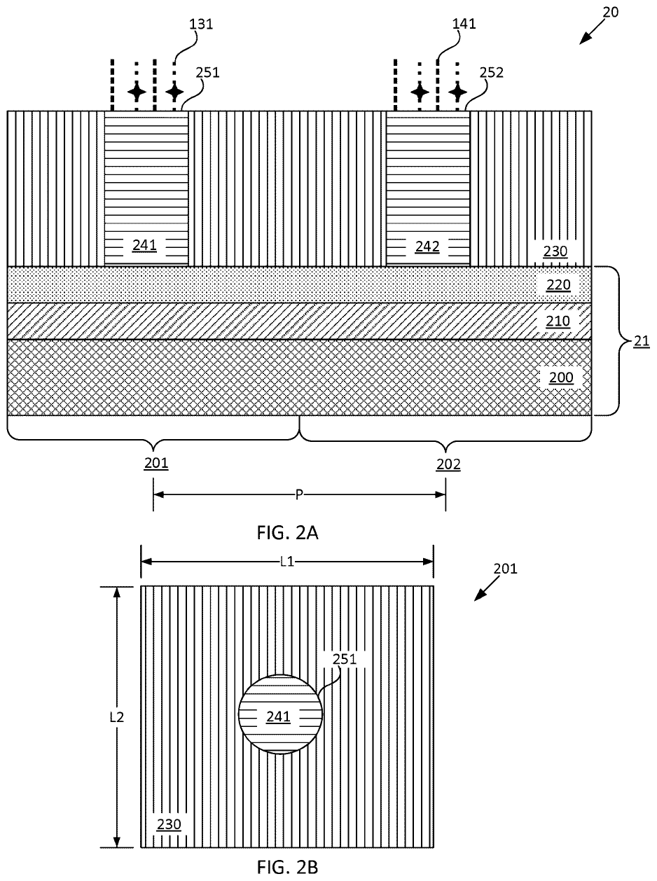

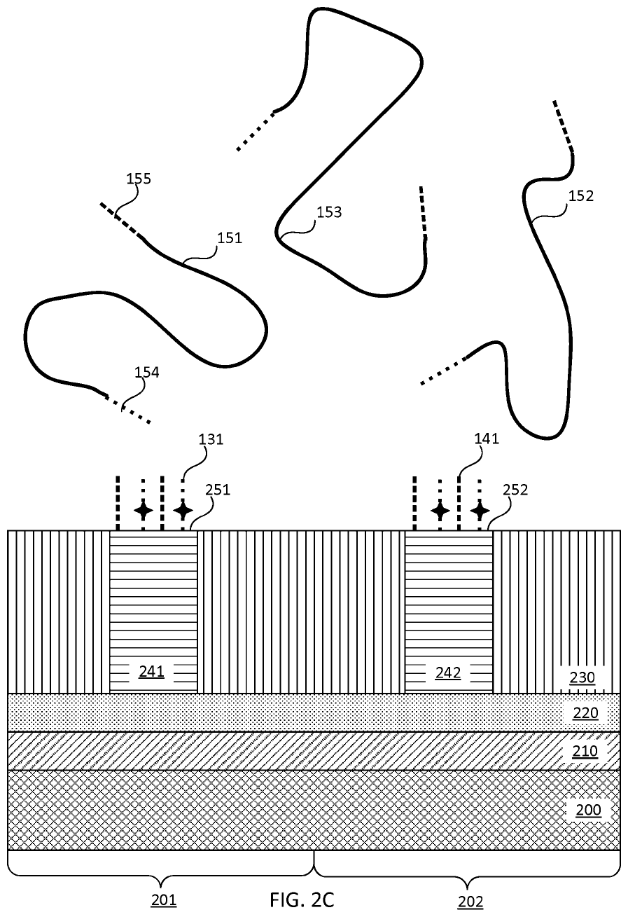

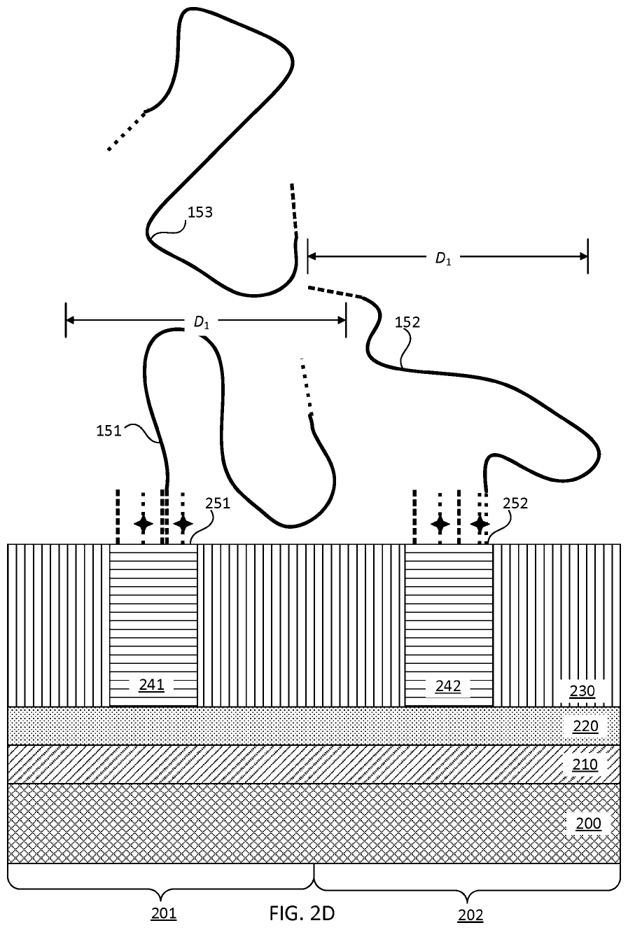

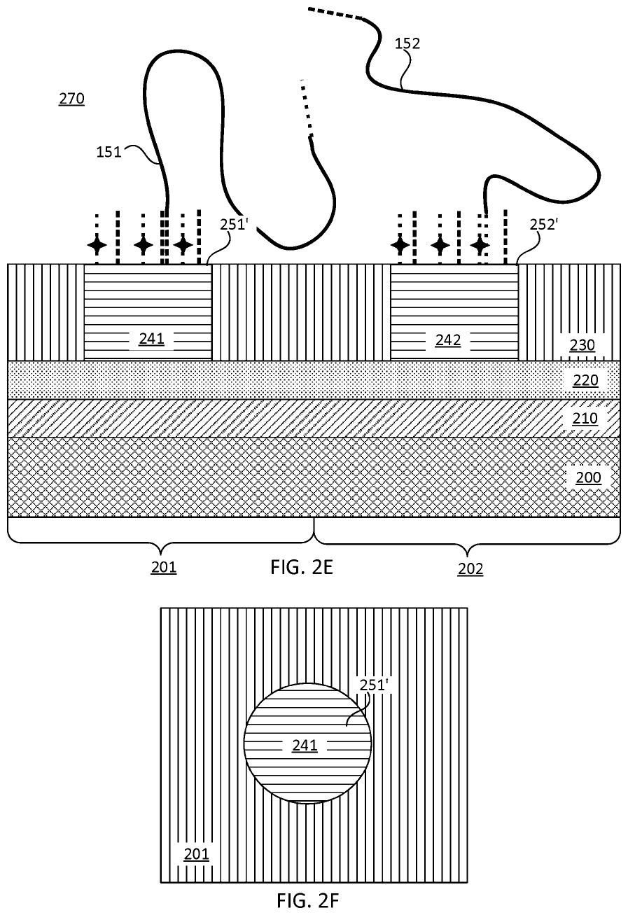

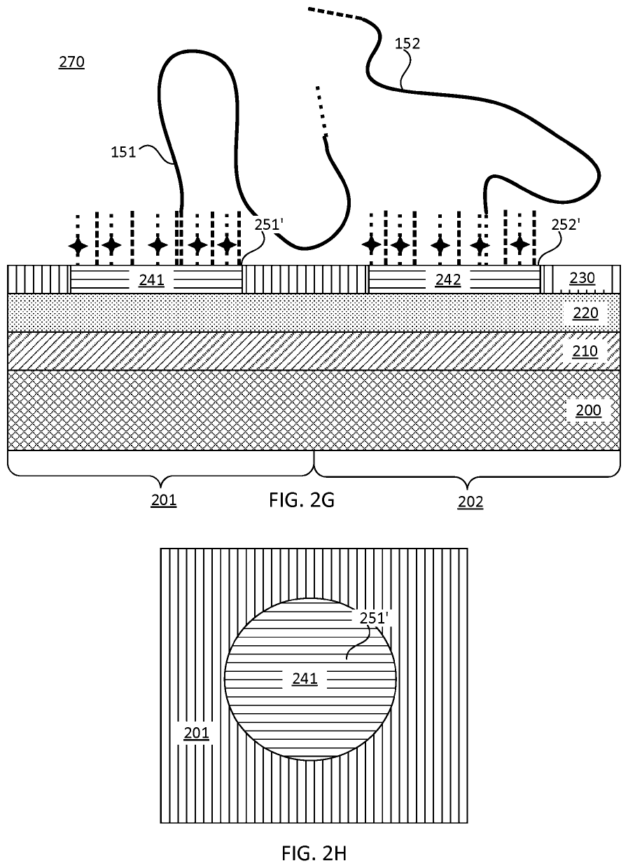

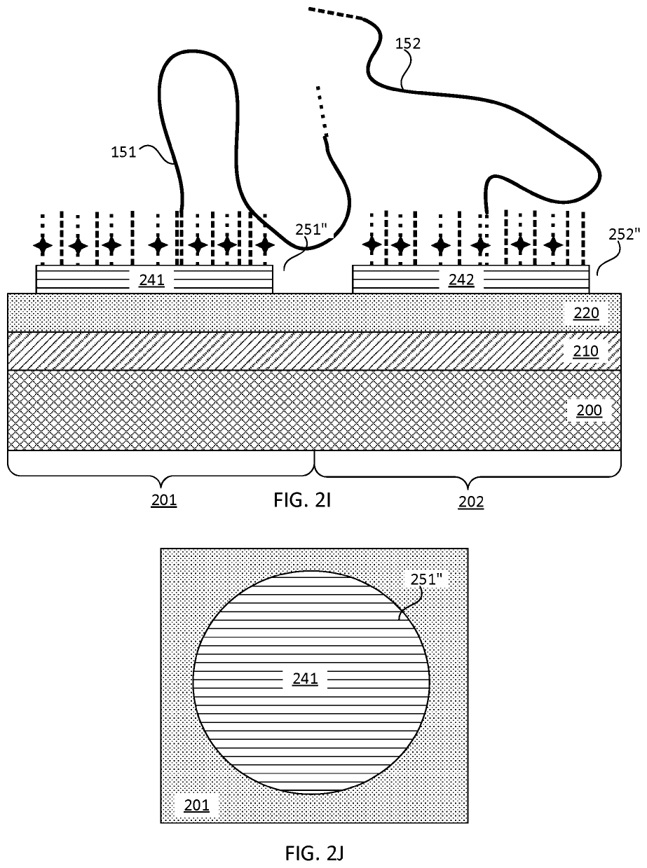

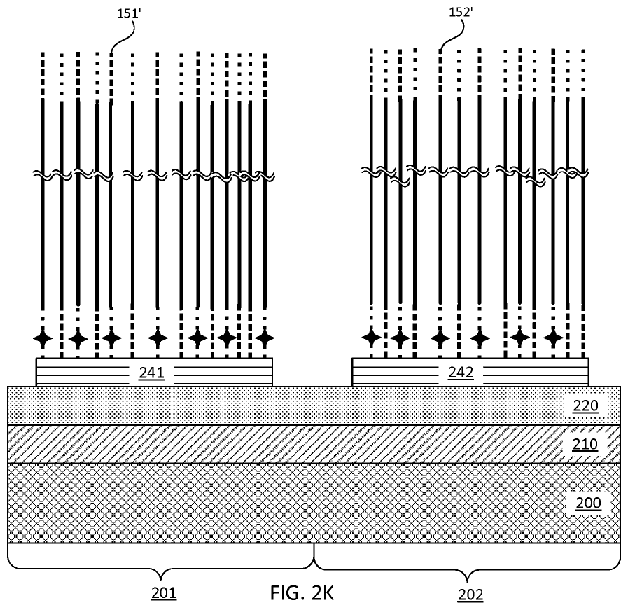

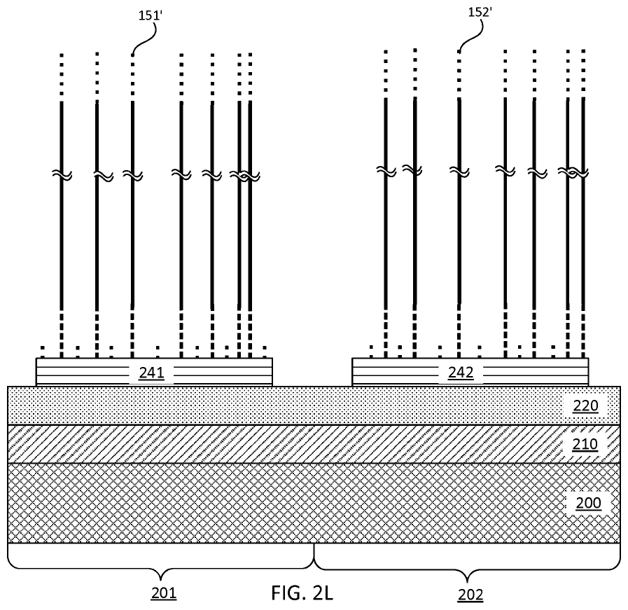

For example, FIGS. 2A-2L schematically illustrate example structures and operations in a process flow for amplifying a polynucleotide on a substrate using a sacrificial material. FIG. 2A illustrates a cross-sectional view of structure 20 that includes substrate 21, sacrificial material 230 disposed on the substrate and having at least one recess 251 therein; and hydrogel 241 disposed within the recess of the sacrificial material. In the nonlimiting example illustrated in FIG. 2A, substrate 21 may include a plurality of different materials which may be located in different layers. Illustratively, substrate 21 optionally may include sensor layer 200 including first and second image sensors 201, 202; passivation layer 210 disposed on sensor layer 200 (e.g., silicon nitride (SiN) or silicon dioxide (glass, SiO₂); and etch stop 220 disposed on passivation layer 210 (e.g., tantalum oxide, TaOₓ). The spacing between image sensors 201, 202 may be referred to as the “pitch” (P) a two-dimensional array of image sensors that includes the image sensors 201, 202 illustrated in FIG. 2A.

Sacrificial material 230 may comprise or be formed of any material that may be preferentially etched relative to etch stop 220, such as silicon dioxide, silicon nitride, or a polymer that can be patterned and/or etched (such as a photoresist), and may have any suitable number of recesses disposed therein. In the nonlimiting example illustrated in FIG. 2A, recess 251 is disposed over (and approximately centered on) first image sensor 201, and recess 252 is disposed over (and approximately centered on) second image sensor 202. For example, FIG. 2B illustrates a plan view of the area corresponding to first image sensor 201, in which it may be seen that within sacrificial material 230, recess 251 is approximately centered relative to the image sensor. It will be appreciated that device 20 may include a sensor circuit that includes an array of any suitable number of image sensors, two of which image sensors are shown in FIG. 2A and one of which image sensors is shown in FIG. 2B. As illustrated in FIG. 2B, image sensor 201 (which may include other circuit components such as an amplifier, that amplify or otherwise condition the electrical signal generated by the image sensor), may have a lateral area L1×L2, where L1 and L2 may be the same as one another or may be different than one another. In some examples, L1 is equal to P, or L2 is equal to P, or both L1 and L2 are equal to P.

Alternatively, substrate 21 may not include any image sensors, e.g., may be or include at least one of a glass, a semiconductor, or a polymer, in which case sacrificial material 230 may comprise or be formed of any suitable material that may be preferentially etched relative to the material(s) of substrate 21 upon which the sacrificial material is disposed.

Hydrogel may be disposed within the recess(es). For example, as illustrated in FIG. 2A, hydrogel 241 may be disposed within recess 251, and hydrogel 242 may be disposed within recess 252. Accordingly, in nonlimiting examples such as illustrated in FIG. 2B, hydrogel 241 disposed in recess 251 is approximately centered relative to the image sensor 201. The hydrogel may include a capture primer for capturing a polynucleotide, and amplification primers for amplifying the polynucleotide. In the nonlimiting example illustrated in FIG. 2A, the hydrogel includes a mixture of amplification primers 131 and 141 (e.g., P5 and P7) that may be used as capture primers to capture a polynucleotide, and also as amplification primers to amplify that polynucleotide in a manner such as will be described with reference to FIGS. 2B-2L. In other examples (not specifically illustrated) the hydrogel further may include a capture primer with a sequence that is orthogonal to that of amplification primers 131 and 141, and that may be used to capture a polynucleotide but not to amplify the polynucleotide. The recess (e.g., recesses) is expandable using an etchant that diffuses through the hydrogel to at least partially remove the sacrificial material substantially without damaging the capture primer or the amplification primers, e.g., in a manner such as will be described with reference to FIGS. 2B-2L.

Turning now to FIG. 2C, structure 20 may be contacted with a fluid, such as an aqueous buffer solution, that includes a plurality of polynucleotides that are to be sequenced or otherwise analyzed. In some examples, the fluid may include cither single-stranded target polynucleotides (as shown), or double-stranded target polynucleotides (not shown), e.g., single- or double-stranded polynucleotide fragments that are generated using commercially available fragmentation or tagmentation techniques, using DNA or RNA that it is desired to sequence. Although only three polynucleotides 151, 152, 153 are illustrated in FIG. 2C, it will be appreciated that the fluid may include thousands, or even millions, of polynucleotides which in some cases may have different lengths than one another. In the nonlimiting example illustrated in FIG. 2C, each of the polynucleotides 151, 152, 153 may include first and second adapters, e.g., first adapter 154 which is substantially complementary to amplification primer 131, e.g., is or includes cP5 (also referred to as P5′); and second adapter 155 which is substantially complementary to amplification primer 141, e.g., is or includes cP7 (also referred to as P7′). In examples in which the polynucleotides are double-stranded, the double-stranded polynucleotides may include an additional single-stranded seeding adapter which is complementary to the above-mentioned orthogonal seeding primer, in a manner such as described in International Patent Application No. PCT/US2022/053002, filed on Dec. 15, 2022 and entitled “Orthogonal Hybridization,” the entire contents of which are incorporated by reference herein.

Referring now to FIG. 2D, adapter 155 of polynucleotide 151 may hybridize to a primer 141 of hydrogel 241 to form a first duplex, and adapter 154 of polynucleotide 152 may hybridize to a primer 131 of hydrogel 242 to form a second duplex. In some examples, the capture of polynucleotide 151 at hydrogel 241 may sterically inhibit other such polynucleotides from being able to become stably coupled to that hydrogel. For example, polynucleotide 151 may occupy a sufficiently large portion of hydrogel 241, such that another polynucleotide may not be able to access that hydrogel in such a manner as to become coupled to that hydrogel. Similarly, the capture of polynucleotide 152 at hydrogel 242 may sterically inhibit other such polynucleotides from being able to become stably coupled to that hydrogel. For example, polynucleotide 152 may occupy a sufficiently large portion of hydrogel 242, such that another polynucleotide may not be able to access that hydrogel in such a manner as to become coupled to that hydrogel.

Illustratively, polynucleotides 151, 152 respectively may have a diameter of gyration having dimension D₁ in the lateral dimension, as shown in FIG. 2D. Within their respective diameters of gyration, polynucleotides 151 and 152 may move (gyrate) in such a manner as to sufficiently occupy all of the space within that diameter that another polynucleotide may not be able to access the respective hydrogel 241 or 242 and thus may not be captured. For example, as illustrated in FIG. 2D, captured polynucleotide 151 may sufficiently occupy the space surrounding hydrogel 241 to inhibit adapters of any other polynucleotides (illustratively, polynucleotide 153) from being captured using a capture primer of hydrogel 241. Similarly, captured polynucleotide 152 may sufficiently occupy the space surrounding hydrogel 242 to inhibit adapters of any other polynucleotides (illustratively, polynucleotide 153) from being captured using a capture primer of hydrogel 242. As such, in some examples, recess 251 and recess 252 may be configured to have a dimension which is approximately equal to or smaller than a diameter of gyration of the polynucleotide. Based on the teachings provided herein, one skilled in the art would be able to design and prepare sacrificial material having a recess with dimension(s) that are approximately equal to or smaller than the diameter of gyration of a polynucleotide with which the sacrificial material and recess are to be used. For example, ssDNA with a length of about 500 base pairs may have a radius of gyration (Rg) of about 16-17 nm (and thus a diameter of gyration of about 32-34 nm) so a recess on the order of about 20-40 nm may be expected to capture a single polynucleotide. As another example, ssDNA with a length of about 100 base pairs may have a Rg of about 8 nm (and thus a diameter of gyration of about 16 nm) so a recess on the order of about 10-20 nm may be expected to capture a single polynucleotide. For further details regarding the radius of gyration of single stranded DNA, see Henrich et al., “Coarse-grained simulation of DNA using LAMMPS: An implementation of the oxDNA model and its applications,” Eur. Phys. J. E Soft Matter 41(5): 57, 16 pages (2018); and Sim et al., “Salt dependence of the radius of gyration and flexibility of single-stranded DNA in solution probed by small angle scattering,” Phys. Rev. E 86:021901 (2012), the entire contents of both of which are incorporated by reference herein.

After capturing the polynucleotide at the recess of the sacrificial material, at least a portion of the sacrificial material may be removed to expand the recess at which the polynucleotide is captured. The etchant may be transported through the hydrogel so as to etch the sidewall(s) forming the recess(es), in addition to etching the top surface, thus expanding the recess(es) in addition to reducing the height of the sacrificial material. For example, as illustrated in FIG. 2E, and in FIG. 2F which shows a cross-section of the area of image sensor 201 in FIG. 2E, the sacrificial material may be exposed to, and is removable using, a liquid or gaseous etchant 270 that is flowed over structure 20. The liquid or gaseous etchant may diffuse (or otherwise be transported through) hydrogels 241 and 242. The etchant suitably may be selected so as to remove the sacrificial material substantially without damaging the capture primer or the amplification primers. Illustratively, in nonlimiting examples in which the sacrificial material 230 is silicon dioxide or silicon nitride, the etchant may include a suitable concentration of citrate (e.g., saline sodium citrate, SSC), tris(hydroxymethyl)aminomethane (“Tris”), carbonate buffer, or HELLMANEX® cleaning concentrate (commercially available from Hellma USA Inc., Plainview NY). In nonlimiting examples in which the sacrificial material 230 is a polymer (such as a photoresist), the etchant may include a photoresist developer, TMAH (tetramethylammonium hydroxide), DMSO (dimethylsulfoxide), or a combination thereof, at any suitable temperature (illustratively, any temperature in the range of room temperature to about 40 °C). At the proper concentration, these example etchants may be expected to be compatible with (e.g., substantially not damage) oligonucleotides, hydrogels, and TaOx.

Etch stop 220 may inhibit damage to the substrate when removing at least the portion of the sacrificial material. For example, etchant 270 may etch the sacrificial material 230 preferentially to etch stop 220, e.g., may etch sacrificial material 220 at a rate that is at least ten times higher, at least twenty times higher, at least 50 times higher, or at least 100 times higher than that of etch stop 220. As illustrated in FIGS. 2E and 2F, partially removing the sacrificial material 230 may expand the diameter of the recess(es) therein. For example, recess 251 expands to form expanded recess 251′ having a larger diameter than recess 251, and recess 252 expands to form expanded recess 252′ having a larger diameter than recess 252.

In some examples, the hydrogel is expandable from the recess into the expanded recess. For example, as illustrated in FIGS. 2E-2F, gravity and/or adhesion between hydrogel 241 and the sidewall forming the recess causes the hydrogel to be retained within the expanded recess. As the lateral area available to the hydrogel increases, the number of amplification primers that are available at the surface of the hydrogel increases, as intended to be represented by additional primers being illustrated in FIG. 2E as compared to FIG. 2D.

Structure 20 may be exposed to the etchant 270 for a sufficient amount of time to expand the recess to a desired diameter. For example, the expanded recesses 251′ and 252′ illustrated in FIGS. 2G and 2H have an even larger diameter than illustrated in FIGS. 2E and 2F following additional exposure to the etchant 270. Indeed, in the nonlimiting example illustrated in FIGS. 21-2J, structure 20 may be exposed to the etchant for a sufficient amount of time to remove the sacrificial material 230 entirely. Despite the removal of sacrificial material 230, adhesion may cause hydrogel 241 to remain within the expanded recess, e.g., disposed over image sensor 201, and may cause hydrogel 242 to remain within the expanded recess, e.g., disposed over image sensor 202.

After expanding the recess, for example through partial or complete removal of the sacrificial material, the captured polynucleotide may be amplified. For example, as illustrated in FIG. 2K, polynucleotides 151, 152 may be amplified in any suitable manner (e.g., using bridge amplification) to obtain amplicons 151′ coupled to hydrogel 241 and amplicons 152′ coupled to hydrogel 242. If amplification operations are repeated until hydrogels 241 and 242 are full, both adapters of the amplicons may not necessarily be hybridized to corresponding amplification primers, and as such the amplicons may extend linearly away from the substrate.

In some examples, certain amplification primers may include non-nucleotide moieties. Such non-nucleotide moieties may include, but are not limited to, excision moieties via which a portion of the capture primers selectively may be removed. For example, amplification primers 131 optionally may include excision moieties 132 and/or amplification primers 141 optionally may include excision moieties (excision moieties of amplification primers 141 not specifically shown in FIGS. 2A-2K). The excision moieties 132 of capture primers 131 may be of the same type as, or a different type than, the excision moieties of capture primers 141. The excision moieties may be located at any suitable position along the length of any suitable primer(s) and may be, but need not necessarily be, the same type of excision moiety as one another. Following amplification of polynucleotides 151, 152, portions of capture primers 131 may be removed by reacting a suitable enzyme or reagent with the respective excision moieties, and/or portions of capture primers 141 may be removed by reacting a suitable enzyme or reagent with the respective excision moieties. The enzyme or reagent used with the excision moieties of capture primers 131 may be the same as, or different than, the enzyme or reagent used with excision moieties of capture primers 141. Excision moieties 132 may be used to remove amplicons that are oriented in a selected direction, so that the remaining amplicons are oriented substantially in the same direction as one another in a manner such as illustrated in FIG. 2L.

In some examples, following the seeding, etching, and amplification operations, the amplicons may be sequenced, e.g., using sequencing-by-synthesis. Regardless of whether the sacrificial material 230 is fully removed as illustrated in FIG. 2K or is only partially removed as illustrated in FIG. 2G, hydrogels 241, 242 and the amplicons 151′, 152′ respectively coupled thereto may be retained within respective expanded recesses. In nonlimiting examples in which the hydrogels are disposed over image sensors, hydrogel 241 and the amplicons 151′ approximately centered over image sensor 201, and hydrogel 242 and amplicons 152′ coupled thereto may be approximately centered over image sensor 202. As such, signals from the respective clusters may couple particularly well into image sensors 201 and 202, for example as compared to signals from a cluster on the sidewall of a recess. Furthermore, and regardless of whether the substrate includes image sensors or not, the clusters may be expected to be at least functionally monoclonal, because the seeding process is performed using hydrogels having a relatively small available area for seeding. In nonlimiting examples, the recess may have a lateral area that is less than about 50% of the lateral area L1×L2 of the image sensor (if used), or less than about 20% of the lateral area of the image sensor, or less than about 10% of the lateral area of the image sensor. Additionally, or alternatively, the recess may have an aspect ratio of about 1:1 to about 4:1 (height:width), e.g., about 2:1 to about 3:1, and/or may have a width of between about 100 nm and about 300 nm. Still further, following such seeding, the sacrificial material allows the cluster area to be expanded so as to provide relatively strong signals during sequencing by synthesis. In some examples, the expanded recess has a lateral area that is at least about 50%, or at least about 60%, or at least about 70%, or at least about 80%, or at least about 90%, or at least about 95% of the lateral area L1×L2 of the image sensor, e.g., about 50% to about 95% of the lateral area of the image sensor. Additionally, or alternatively, the expanded recess may have an aspect ratio of at least about 1:2 to about 1:100 (height:width), e.g., about 1:2 to about 1:10, and/or may have a width of between about 350 nm to about 5 μm, e.g., about 500 nm to about 1000 nm, or about 1 μm to about 5 μm, or about 350 nm to about 2 μm.

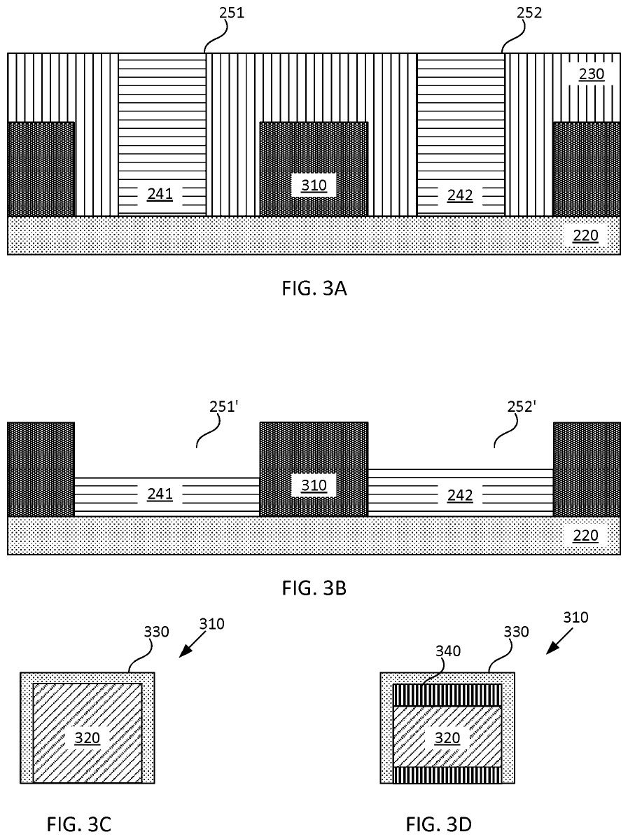

Although FIGS. 2A-2L illustrate an example in which only sacrificial material 230 is disposed on substrate 21, it will be appreciated that in other examples, other material(s) may be disposed on the substrate. Such other material(s) optionally may be used to define the area which the hydrogel occupies following removal of the sacrificial material, and/or to perform other functions. For example, FIGS. 3A-3D illustrate example structures that may be included in the structures described with reference to FIGS. 2A-2L. FIG. 3A is a simplified illustration of structure 20 in which passivation layer 210, sensor layer 200, and amplification primers are omitted for simplicity; in other examples, the substrate may not include an image sensor, e.g., may be or include at least one of a glass, a semiconductor, or a polymer. As illustrated in FIG. 3A, structure 310 may be disposed on substrate 20, e.g., may be disposed on etch stop layer 220 between recess 251 and recess 252, or disposed on other suitable substrate. Sacrificial material 230 may be disposed over structure 310, and may be removed preferentially to structure 310. For example, as illustrated in FIG. 3B, structure 310 may remain on the etch stop layer 220 after sacrificial material 230 is partially or completely removed. In some examples, structure 20 may include a plurality of structures 310, and structures 310 may define expanded recesses 251′ and 252′ after sacrificial material 230 is completely removed, e.g., in a manner similar to that of partially removed sacrificial material 230 as illustrated in FIGS. 2G-2H.

Structure 310 may include any suitable material or combination of materials. For example, structure 310 may include a single material that is substantially not removed using etchant 270, illustratively, the same material that forms etch stop layer 220. Alternatively, structure 310 may include a combination of materials. For example, FIG. 3C illustrates structure 310 that includes first material 320 that potentially would be removable using etchant 270 (such as patterned SiO₂ or SiN or other passivation material), but instead is protected by an overlayer of second material 330 that is substantially not removed using etchant 270, illustratively, the same material that forms etch stop layer 220. As such, first material 320 is not removed (because it is protected by second material 330). As another example, FIG. 3D illustrates structure 310 that includes first material 320 that potentially would be removable using etchant 270 (such as glass or other passivation material), one or more additional layers of material 340 that potentially could be removed using etchant 270 (such as glass or other passivation material) or that may not be removable using etchant 270 (such as tantalum), and is protected by an overlayer of second material 330 that is substantially not removed using etchant 270, illustratively, the same material that forms etch stop layer 220. As such, structure 310 is not removed (because it is protected by second material 330). In some examples, material 340 serves as a light shield and would also not be removed by 270 (for example because it is protected by second material 330). In some examples, material 340 may be a metal such as Ti, W, Al, Cu, Ta, or other suitable conductor. This light shield prevents or inhibits unwanted ambient or excitation light to irradiate the image sensor in such a manner that would interfere with imaging of the desired signal. With such a light shield, light may substantially only pass through the recess (e.g., nano well) and underlying optical filter stack where unwanted photons get filtered out. Material 330 illustrated in FIGS. 3C and 3D thus form a second etch stop disposed on the substrate, the second etch stop substantially surrounding the recess. The second etch stop may define a lateral area of an expanded recess formed by the at least partial removal of the sacrificial material.

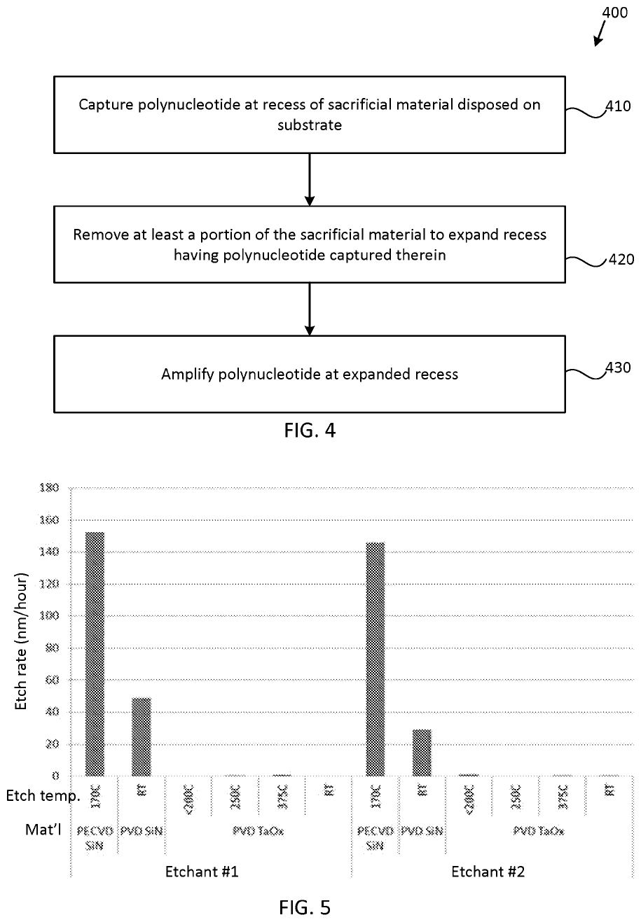

FIG. 4 illustrates an example flow of operations in a method 400 for amplifying a polynucleotide using a sacrificial material. Although method 400 may be implemented using structure 20 described with reference to FIGS. 2A-2L or the structures described with reference to FIGS. 3A-3D, method 400 may be implemented using any other suitable structure. Method 400 may include capturing a polynucleotide at a recess of a sacrificial material disposed on a substrate (operation 410), e.g., in a manner such as described with reference to FIGS. 2A-2D. Method 400 also may include removing at least a portion of the sacrificial material to expand the recess at which the polynucleotide is captured (operation 410), e.g., in a manner such as described with reference to FIGS. 2E-2J. Method 400 also may include amplifying the polynucleotide at the expanded recess (operation 420), e.g., in a manner such as described with reference to FIGS. 2K-2L.

In some examples, a method for sequencing a polynucleotide may include capturing and amplifying the polynucleotide using method 400, and sequencing the amplified polynucleotide within the expanded recess. In some examples, a method for sequencing a polynucleotide may include capturing and amplifying the polynucleotide using structure 20; and sequencing the amplified polynucleotide within the expanded recess. In some examples, a kit may include structure 20, and an etchant for at least partially removing the sacrificial material.

From the foregoing disclosure, it will be appreciated that primers and adapters having any suitable sequences may be used. In one nonlimiting example, amplification primers 131 are P5 amplification primers, and amplification primers 141 are P7 amplification primers. P5 amplification primers, which are commercially available from Illumina, Inc. (San Diego, CA) have the sequence 5′-AATGATACGGCGACCACCGA-3′ (SEQ ID NO: 1). P7 amplification primers, which also are commercially available from Illumina, Inc., have the sequence 5′-CAAGCAGAAGACGGCATACGA-3′ (SEQ ID NO: 2). Adapters 154 may be full-length complementary P5 adapters (cP5) having the sequence 5′-TCGGTGGTCGCCGTATCATT-3′ (SEQ ID NO: 3), and are commercially available from Illumina, Inc. Adapters 155 may be full-length complementary P7 adapters (cP7) having the sequence 5′-TCGTATGCCGTCTTCTGCTTG-3′ (SEQ ID NO: 4), and are commercially available from Illumina, Inc.

It will be appreciated that various examples herein may be used with operations consistent with “bridge amplification” or “surface-bound polymerase chain reaction” and/or with other amplification modalities. One such amplification modality is “exclusion amplification,” or ExAmp. One such amplification modality is “exclusion amplification,” or ExAmp. Exclusion amplification methods may allow for the amplification of a single target polynucleotide per substrate region and the production of a substantially monoclonal population of amplicons in a substrate region. For example, the rate of amplification of the first captured target polynucleotide within a substrate region may be more rapid relative to much slower rates of transport and capture of target polynucleotides at the substrate region. As such, the first target polynucleotide captured in a substrate region may be amplified rapidly and fill the entire substrate region, thus inhibiting the capture of additional target polynucleotide in the same substrate region. Alternatively, if a second target polynucleotide attaches to same substrate region after the first polynucleotide, the relatively rapid amplification of the first polynucleotide may fill enough of the substrate region to result in a signal that is sufficiently strong to perform sequencing by synthesis (e.g., the substrate region may be at least functionally monoclonal). The use of exclusion amplification may also result in super-Poisson distributions of monoclonal substrate regions; that is, the fraction of substrate regions in an array that are functionally monoclonal may exceed the fraction predicted by the Poisson distribution.

Increasing super-Poisson distributions of useful clusters is useful because more functionally monoclonal substrate regions may result in higher quality signal, and thus improved SBS; however, the seeding of target polynucleotides into substrate regions may follow a spatial Poisson distribution, where the trade-off for increasing the number of occupied substrate regions is increasing the number of polyclonal substrate regions. One method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present compositions and methods may be adapted for use with recombinase to facilitate the invasion of the present capture primers into the present target polynucleotides when the recombinase mediates a sequence match. Indeed, the present structures and methods may be adapted for use with any surface-based polynucleotide amplification methods such as thermal PCR, chemically denatured PCR, and enzymatically mediated methods (which may also be referred to as recombinase polymerase amplification (RPA) or ExAmp).

For still further examples of amplification methods that are compatible with the present structures and methods, see International Patent Application No. PCT/US2022/053005 to Ma et al., filed Dec. 15, 2022 and entitled “Hybrid Clustering;” U.S. patent application Ser. No. 18/193,480 to Ma et al., filed Mar. 30, 2023 and entitled “Paired-End Resynthesis Using Blocked P5 Primers;” and International Patent Application No. PCT/US2022/053002, filed on Dec. 15, 2022 and entitled “Orthogonal Hybridization,” the entire contents of each which are incorporated by reference herein.

### Working Examples

The following examples are intended to be purely illustrative, and not limiting in any way.

FIG. 5 illustrates etch rates measured for different materials under different conditions. More specifically, samples were prepared that respectively included the following materials (“Mat'1” in FIG. 5): TaOₓ formed using physical vapor deposition (PVD TaOx), silicon nitride (SiN) formed using PVD (PVD SiN), and SiN formed using plasma-enhanced chemical vapor deposition (PECVD SiN). The samples were exposed to an etchant which was either etchant #1 (including 1 M Tris) or etchant #2 (including 0.5 M Tris). As shown in FIG. 5, the PECVD SiN was observed to have an etch rate of about 150 nm/hour when exposed to etchant #1 at a temperature of about 170 °C, and an etch rate of about 140 nm/hour when exposed to etchant #2 at a temperature of 170 °C. The PVD SIN was observed to have an etch rate of about 50 nm/hour when exposed to etchant #1 at room temperature (RT), and an etch rate of about 30 nm/hour when exposed to etchant #2 at room temperature. The PVD TaOx was observed to have etch rates of about 2 nm/hour or less when exposed to etchant #1 at temperatures of less than 200 °C, 250 °C, 375 °C, and room temperature, and similarly was observed to have etch rates of about 2 nm/hour or less when exposed to etchant #2 at temperatures of less than 200 °C, 250 °C, 375 °C, and room temperature. From these results, it may be understood that PVD TaOx has a significantly lower etch rate in etchant #1 or etchant #2 under a variety of conditions than does PVD SiN or PECVD SiN. Accordingly, it may be further understood that SiN, and other materials with etch rates that are significantly higher than that of TaOx (e.g., at least ten times higher, at least twenty times higher, at least 50 times higher, or at least 100 times higher) suitably may be used as a sacrificial material in structures and operations such as described with reference to FIGS. 2A-2L, 3A-3D, and 4. Further, it may be understood that TaOx suitably may be used as an etch stop to inhibit damage to underlying materials, e.g., is substantially not removed by at least some etchants that remove sacrificial materials such as silicon nitride.

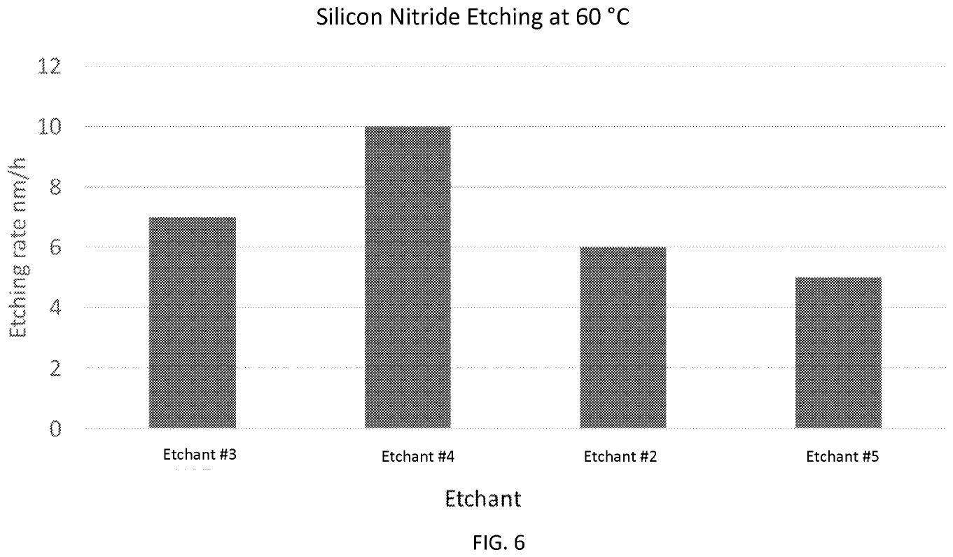

FIG. 6 illustrates etch rates measured for silicon nitride at 60 °C using different etchants. More specifically, silicon nitride formed using PECVD was exposed to etchant #2, etchant #3 (including 5×SSC with tween 0.1%), etchant #4 (AZ® 400k 1:4), and etchant #5 (including 1.0 M Tris) at 60 °C. AZ® 400k is a photoresist developer based on buffered KOH and commercially available from Microchemicals GmbH, Ulm, Germany. The silicon nitride was observed to have an etch rate of about 7 nm/hour when exposed to etchant #3, an etch rate of about 10 nm/hour when exposed to etchant #4, an etch rate of about 6 nm/hour when exposed to etchant #2, and an etch rate of about 5 nm/hour when exposed to etchant #5. From these results, it may be understood that a sacrificial material, such as silicon nitride, suitably may be etched at a temperature which is safe for hydrogel and primers.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of amplifying a polynucleotide, the method comprising:
capturing the polynucleotide at a recess of a sacrificial material disposed on a substrate;
removing at least a portion of the sacrificial material to expand the recess at which the polynucleotide is captured; and
amplifying the polynucleotide at the expanded recess.

2. The method of claim 1, wherein a hydrogel is disposed within the recess of the sacrificial material, the hydrogel comprising:
a capture primer to which the polynucleotide hybridizes when capturing the polynucleotide; and
amplification primers to which amplicons of the polynucleotide hybridize when amplifying the polynucleotide.

3. The method of claim 2, wherein the hydrogel expands from the recess into the expanded recess.

4. The method of claim 1, wherein only a portion of the sacrificial material is removed.

5. The method of claim 1, wherein the sacrificial material is entirely removed.

6. The method of claim 1, wherein the sacrificial material comprises silicon dioxide, silicon nitride, or a polymer.

7. The method of claim 1, wherein the sacrificial material is removed using an etchant.

8. (canceled)

9. The method of claim 1, wherein the substrate comprises a first etch stop on which the sacrificial material is disposed, the first etch stop inhibiting damage to the substrate when removing at least the portion of the sacrificial material.

10. (canceled)

11. The method of claim 1, wherein a second etch stop is disposed on the substrate, the second etch stop substantially surrounding the recess.

12. The method of claim 11, wherein the second etch stop defines a lateral area of the expanded recess.

13. The method of claim 11, wherein the second etch stop comprises tantalum oxide or tantalum.

14. (canceled)

15. The method of claim 1, wherein the substrate comprises at least one of a glass, a semiconductor, or a polymer.

16. The method of claim 1, wherein the substrate does not include any image sensor.

17. The method of claim 1, wherein the substrate comprises an image sensor having a lateral area.

18. The method of claim 17, wherein the recess has a lateral area that is less than about 50% of the lateral area of the image sensor.

19. (canceled)

20. The method of claim 17, wherein a single recess is disposed over the image sensor.

21. (canceled)

22. The method of claim 1, wherein the recess has an aspect ratio of about 1:1 to about 4:1 (height:width).

23. The method of claim 1, wherein the expanded recess has an aspect ratio of at least about 1:2 to about 1:100 (height:width).

24-25. (canceled)

26. A method for sequencing a polynucleotide, the method comprising:
capturing and amplifying the polynucleotide using the method of claim 1; and
sequencing the amplified polynucleotide at the expanded recess.

27. A structure, comprising:
a substrate;
a sacrificial material disposed on the substrate and having a recess therein; and
a hydrogel disposed within the recess of the sacrificial material, the hydrogel comprising:
a capture primer for capturing a polynucleotide; and
amplification primers for amplifying the polynucleotide,
wherein the recess is expandable using an etchant that diffuses through the hydrogel to at least partially remove the sacrificial material, substantially without damaging the capture primer or the amplification primers.

28-50. (canceled)


SEQUENCE LISTING (4 sequences)

SEQ ID NO: 1 - DNA, 20 bp, synthetic construct
  source 1..20: mol_type = other DNA; note = P5 amplification primer
aatgatacggcgaccaccga

SEQ ID NO: 2 - DNA, 21 bp, synthetic construct
  source 1..21: mol_type = other DNA; note = P7 amplification primer
caagcagaagacggcatacga

SEQ ID NO: 3 - DNA, 20 bp, synthetic construct
  source 1..20: mol_type = other DNA; note = complementary P5 adapter
tcggtggtcgccgtatcatt

SEQ ID NO: 4 - DNA, 21 bp, synthetic construct
  source 1..21: mol_type = other DNA; note = complementary P7 adapter
tcgtatgccgtcttctgcttg